(19) United States
(12) Patent Application Publication
(10) Pub. No.: US 2010/0098784 A1
(43) Pub. Date: Apr. 22, 2010

(54) CAPSULE CONTAINING PLANT PREPARATIONS

(75) Inventor: Dieter Wolfgang Engel, Zuzwil (CH)

Correspondence Address: THE NATH LAW GROUP, 112 South West Street, Alexandria, VA, 22314, US

(73) Assignee: Swiss Caps Rechte und Lizenzen AG, Kirchberg (CH)

(21) Appl. No.: 12/450,440

(22) PCT Filed: Mar. 7, 2008
(86) PCT No.: PCT/EP2008/052744

§ 371 Date: Sep. 25, 2009

(30) Foreign Application Priority Data
Mar. 26, 2007 (CH) 00483/07

(51) Int. Cl. A61K 36/185; A61P 37/06

(52) U.S. Cl. 424/725

(57) ABSTRACT

The invention relates to a composition containing a lipophilic extract of Vitellaria paradoxa and at least one amino sugar or a pharmaceutically acceptable salt thereof, in addition to a capsule containing said composition. The composition is used for the prophylaxis and treatment of inflammatory osteoarthritis.

The present invention relates to capsules containing a combination of pharmacologically active substances from Vitellaria paradoxa extracts and amino sugar derivatives, their production and use for the prophylaxis and treatment of inflammatory degenerative arthropathies.

Glucosamine and salts thereof, and chondroitin and salts thereof, have long been proposed for dietetic treatment and prevention for joint problems. This is understandable against the background that cartilage and synovial fluid mainly consist of mucopolysaccharides, which in their turn consist of the two most important acetylated amino sugars N-acetylglucosamine and N-acetylgalactosamine.

These amino sugars are obtained from special types of tissues of vertebrates or from chitin of crustacea and, for application in humans, are administered as hydrochlorides or sulfate salts in tablets and capsules. Amino sugars and their acetylates are highly water-soluble. Administration in the form of a soft capsule as a rule requires the use of high proportions of mono- and di- or triglycerides and waxes (beeswax), in order to obtain a flowable, sufficiently lipophilic mass, which do not react with the capsule shell containing water during capsule filling with discoloration or embrittlement.

However, a consequence of this is that conventional formulations with amino sugars as pharmacological ingredient are relatively voluminous, with a substantial proportion of the volume being taken up by excipient without pharmacological activity.

The karite tree, also called shea butter tree, is a tropical tree with height of 10-15 m, which is mainly indigenous to the Sudan and Niger. The plum-shaped fruits of the karite tree, belonging to the Sapotaceae family (botanical name: Vitellaria paradoxa; alternative botanical name: Butyrospermium parkii) contain as seeds kernels (nuts), which have high fat content (Wissebach, H. (1969), Pflanzen- und Tierfette, Handbuch der Lebensmittelchemie (Plant and animal fats, Manual of food chemistry), K. G. D. Acker. L.; Bergner, W.; Heimann, W.; Kiermeir, F.; Schormüller, J.; Souci, S. W., Springer Verlag Berlin. IV Fette und Lipoide (fats and lipids: 41 ff.).

By breaking open the nuts and hot extraction, after refining (bleaching, deodorizing) a fat can be obtained for food purposes and for cosmetics that is traded globally, and has now in many cases replaced the considerably more expensive cocoa butter. As a rule this shea butter contains 3-15% unsaponifiable constituents, comprising approx. 70% hydrocarbons, 7% squalene, 7% triterpene alcohols, and 8% phytosterols (Massera, A. M. F., E.; Proserpio, G. Rivista italiana Essenze Profumi 60 (7) (1978): 414-421; Nowak, G. A. (1984). Die kosmetischen Präparate (Cosmetic preparations), Verlag für Chem. Industrie H. Zielkowsky K G, Augsburg). Shea butter has also already been used for reducing the metabolically utilizable proportion of fat in food (U.S. Pat. No. 6,149,961).

The pharmaceutical use of leaves or of extracts from the seeds of the karite tree is already known from traditional medicine. The carriers of the pharmacological action in Vitellaria extracts have not yet been identified in detail, however, although interesting substances were noted some time ago (Somorin, O., “Preliminary studies on the physiological effects of extracts from the roots of Shea butter tree.” J. Clin. Pharmacol. 13: 178 (1973); Di Vincenzo, D. M., Steve; Serraiocco, Arnaldo; Vito, Raffaella; Wiesman, Zeev; Bianchi, Giorgio. “Regional variation in shea butter lipid and triterpene composition in four African countries.”, Journal of Agricultural and Food Chemistry 53 (19) (2005): 7473-7479). Possibly they are phytosterols and polycyclic triterpene alcohols and their acetic and cinnamic acid esters with pronounced antiinflammatory, and/or antiarthritic action.

WO 2004/002504 describes the isolation of triterpenoids or saponins from constituents of the karite tree, which were made water-soluble by saponification, and the production of tablets containing these substances for the reduction of serum cholesterol and for the treatment of inflammatory diseases.

Semisolid cosmetic or pharmaceutical preparations often employ shea butter as a viscosity-increasing excipient, without the pharmacological active substances possibly present in shea butter being utilized (see e.g. EP-1 392 335). For this application the shea butter is highly purified and refined. During this processing, most of the pharmacologically active ingredients are separated and discarded. The highly pure shea butter that remains no longer possesses any pharmacological activity.

However, the formulation of lipophilic Vitellaria extracts in solid form and in particular in tablet form basically for technical reasons can only be achieved with great difficulty or not at all.

The problem underlying the present invention was to provide a dosage form that is as compact as possible, which contains amino sugars as pharmacological ingredient and in addition has a greater pharmacological action than conventional preparations based on amino sugars.

Surprisingly, it was found according to the present invention that the problem stated above is solved when a lipophilic Vitellaria extract is used for the production of a pharmaceutical capsule preparation containing amino sugars.

This can be attributed primarily to the following reasons:
by combining liquid Vitellaria extracts with solid amino sugars and their salts, the dosage form can be kept much smaller and thus more space-saving, as the active substance Vitellaria extract is simultaneously the “carrier matrix” for the solid amino sugar compounds.
in a capsule, it is possible to use pharmacologically active lipophilic liquid Vitellaria extracts, which can only be achieved with difficulty in other solid oral dosage forms.
the lipophilic liquid Vitellaria extracts can be prepared as capsule filling with emulsifiers and solubilizers, so that they mix rapidly with aqueous biological media such as saliva, gastric juice or the intestinal milieu or can be emulsified in these. As a result their pharmacological activity is increased.
in a capsule, appropriately prepared lipophilic Vitellaria extracts can be provided in liquid, emulsified or dispersed form.

It is therefore possible for all carriers of the pharmacological activity of Vitellaria species (hydrophilic and lipophilic components) and amino sugars to be provided in combination in a capsule. For the reasons stated above this is not possible for example in tablets.

The present invention does not use any pharmacologically inactive excipients for the amino sugars, but instead Vitellaria extracts, which themselves have a corresponding pharmacological activity, which supplements the activity of the amino sugars synergistically. In contrast to the conventional voluminous carrier matrixes for amino sugars, the Vitellaria extract used according to the invention is compact, pharmacologically active and does not represent “dead” volume.

The present invention therefore also relates to a composition containing a lipophilic extract from Vitellaria paradoxa and at least one amino sugar or a pharmaceutically acceptable salt thereof. This composition is suitable in particular as filling for capsules.

Capsules are established dosage forms for medicinal products and food supplements. They are formed as a core and shell structure, i.e. an ingredient of any consistency (the core) is surrounded by a shell of suitable shell material. In particular, a distinction is made between hard capsules and soft capsules.

In the case of hard capsules, the shell material consists of a rather thin film (with a thickness of up to 200 μm), which is nevertheless dimensionally stable. As a rule the shell is made up of two complementary parts that can be joined together (body and cap). The finished capsule is obtained by filling one part and joining the two parts together. Gelatin, cellulose derivatives, gums, PVA (polyvinyl alcohol), PVP (polyvinylpyrrolidone) and other synthetic and natural polymers or mixtures of polymers with other substances, are usually used as film material. Hard capsules are as a rule filled with pulverulent, or particulate (pellets) fillers, but can also be filled with liquid or pastelike fillers.

In the case of soft capsules, as a rule the shell is thicker (with a thickness greater than 200 μm). The shell material is selected from the group comprising gelatin, starch, gums (hydrophilic biopolymers such as carrageenan, guar, alginate etc.) or other natural or synthetic polymers or mixtures thereof and as a rule contains a plasticizer. Owing to the production process, soft capsules as a rule consist of single-part films (in the case of production by a drop, ring-extrusion, injection molding, co-injection molding process) or of two films that are heat-sealed together (in the case of the rotary die process). Soft capsules are as a rule filled with liquid or pastelike fillers, but can also be filled with pulverulent or particulate (pellets) fillers.

Preferably, as interactions barely occur between hydrophilic shell and filler, lipophilic liquid fillers are alone or as carrier matrix for a suspension of crystalline water-soluble substances in a lipophilic matrix. Melts of lipophilic substances hardened by solidification and highly viscous suspensions of water-soluble particles in a lipophilic matrix are also suitable for filling hard capsules or for filling soft capsules whose shell is produced by melt-extrusion technology. This is described in EP-1 103 254 and EP-1 586 436, whose contents in this respect are expressly referred to.

Capsules and methods for their production are adequately known (e.g. Stanley, J. P. “Soft gelatin capsules” in: Lachman et al. (Eds.) “The theory and practice of industrial pharmacy”, Philadelphia, Lea & Febiger, 3rd edition (1986); Hofer et al. in: Fahrig, W.; Hofer, U. (Eds.) “Die Kapsel” (The capsule), Wiss. Verlagsgesellschaft mbH, Stuttgart; Paperback APV Vol. 7, 1st edition, 1981; Gennadios A., “Soft gelatin capsules”, in Gennadios A. Ed., Protein-based films and coatings, CRC Press, Boca Raton, 2002, ISBN 1-58716-107-9).

The lipophilic Vitellaria extracts, which can be used in the composition according to the present invention, are preferably obtained from the above-ground parts of the Vitellaria plant, e.g. from flowers, fruits, leaves or bark. The extract from the berries, especially from the seeds, the so-called shea nuts, is especially preferred. According to the invention, the extracts can be obtained from Vitellaria species that are selected from the group comprising Vitellaria Gaertn.f., Vitellaria campechiana, Vitellaria mammosa (L.), Vitellaria nervosa (DC.), Vitellaria paradoxa Gaertn., Vitellaria paradoxa subsp. Nilotica.

Extraction from the appropriate parts of the shea butter tree takes place in a manner known by a person skilled in the art. Depending on whether hydrophilic or lipophilic constituents are to be extracted, the plant parts are treated with an appropriate solvent. In the case of hydrophilic extracts, water or a polar solvent, for example methanol or ethanol, can be used. In the case of lipophilic extracts, correspondingly apolar solvents such as hydrocarbons can be used. Of course, corresponding polar or apolar solvent mixtures can also be used in combination or successively.

The extraction conditions are familiar to a person skilled in the art. Extraction can be carried out at room temperature. Often, however, it is preferable to carry out extraction at elevated temperatures (up to the boiling point of the respective solvent or solvent mixture), for example in Soxhlet apparatus. Optionally the extraction can be carried out several times (also with different solvents).

The extract obtained in this way can optionally undergo further purification steps or separation steps.

Extracts are to be understood as all extracts from plant material of any plant constituents with partial omission of primary plant ingredients such as cellulose, saccharides, proteins, and triglycerides. The purpose of these extractions is the enrichment of certain secondary plant ingredients, in particular those with desirable pharmacological properties.

In particular, lipophilic extracts can be obtained by extracting the naturally present oils/fats or fat-soluble fractions in the plant tissue. During this, the naturally present triglycerides/waxes/hydrocarbons can also perform the role of extractant. Owing to insufficient volatility these cannot be evaporated, but for further enrichment of secondary plant ingredients they are to be treated again with a solvent or by methods such as countercurrent extraction, supercritical extraction, chromatographic methods, (steam or vacuum) distillation or other separation processes.

Lipophilic extracts can also be obtained directly by extraction with a lipophilic low-molecular solvent such as high-proof ethanol, acetone, ethyl acetate, hexane, chloroform etc. or supercritical carbon dioxide, which can be evaporated. The term “lipophilic” describes, technically, substances with a partition coefficient (log p) in octane/water or octane/common salt solution greater than 1.

Comminution of the plant materials, disruption of the plant cells by grinding, heating, ultrasonic treatment, and extraction, enrichment by evaporation of the solvent and purification are sufficiently familiar to a person skilled in the art.

Similarly, water-soluble extracts can be obtained by extracting with more-hydrophilic solvents or by chemical reaction (e.g. saponification of the secondary plant ingredients) of lipophilically extracted fractions.

The term “amino sugar” is a collective term for monosaccharides in which a hydroxyl group has been replaced with an amino group. This can be a primary amino group, but also a secondary or tertiary amino group. As examples we may mention glucosamine, preferably D-glucosamine, galactosamine, preferably D-galactosamine, chondroitin or neuraminic acid and the corresponding N-acetylated derivatives N-acetylglucosamine and N-acetylgalactosamine, hyaluronic acid, dermatan, keratan, heparin and pharmaceutically acceptable salts thereof. In the composition according to the present invention, the amino sugars can be in free form or in the form of their pharmaceutically acceptable salts (for example the corresponding halides or sulfates).

Based on their antiinflammatory and antidegenerative activity, Vitellaria extracts can be used in the area of prevention and treatment of rheumatic diseases. The rheumatic diseases include in particular: polyarthritis of unknown origin (in particular rheumatoid arthritis) and osteoarthritis (osteoarthrosis). According to the invention it is therefore especially appropriate to provide the antiinflammatory action of Vitellaria extracts together with amino sugars for the biogenic formation of mucopolysaccharides in the joints.

For this it is preferable to design the capsules so that a minimum possible number of capsules required for the dosage provides the maximum possible bioavailability. This is achieved by suspending the amino sugars in a matrix of lipophilic Vitellaria extracts and/or fatty oils from Vitellaria that is pumpable at 25-35° C. and does not form sediment on standing.

The Vitellaria extract can be adjusted, depending on the proportion of unsaponifiable constituents, or by the proportion of C18:0 fatty acids in the triglyceride fraction, to a solidification or melting range of 20-45° C. In particular, the proportion of lower or higher melting fractions can be controlled by destearinization (winterization). Especially preferably, the extract is adjusted to a drop point of 25 to 40° C., more preferably for soft gelatin capsules to a drop point of 20-30° C., and for hard capsules, starch capsules and capsules produced by the melt-extrusion process to a drop point of 30-45° C.

According to a preferred embodiment the capsules according to the invention are therefore formulated with a filling of Vitellaria extract or fatty oils from Vitellaria and with amino sugars or their salts, so that a filling that is pumpable at temperatures above +40° C., and solidifies at temperatures below +35° C., is filled in suitable capsules.

According to another embodiment of the present invention, the capsule for oral administration can be designed so that on contact with the gastric juice it is emulsified, forming an emulsion. Such capsules are known in principle (e.g. EP-0 637 715, C. W. Pouton, Advanced Drug Delivery Reviews, 25: 47-58 (1997)).

For this, one or more emulsifiers can be added or the lipid matrix can be saponified to free fatty acids or soaps (fatty acid-alkali salt) (by means of mono- and distearates) or the extracts can be converted to the form of partially saponified (amphoteric) extracts (containing unsaponifiable components, mono- and diglycerides and fatty acid-alkali salts). Emulsifiers and solubilizers that can be used according to the invention are well known by a person skilled in the art and do not need further explanation here. Purely as examples we may mention, as solubilizers/co-solvents: octanoic acid monoglyceride, free fatty acids or fatty acid esters (e.g. oleic acid, fish-oil ethyl esters (in various forms). Examples of emulsifiers are: lecithin, Span (sorbitan fatty acid ester), Tweens (polysorbates), polyglycerol esters.

The amounts of Vitellaria extract and amino sugar contained in the composition are not important. However, according to the invention it is preferable to use Vitellaria extract and amino sugar in a ratio from 60:40 to 40:60.

The composition according to the invention can in addition contain other active substances selected from the group comprising cysteine, taurine and salts thereof, glutathione and salts thereof, glycine, glutamic acid, tocopherols, tocotrienols, rosemary extract or its constituents, carnosine and rosemary acid.

The present invention is explained in more detail below on the basis of nonlimiting examples.

### EXAMPLE 1

### Extract Prepared From Crushed Kernels (Nuts) of Vitellaria paradoxa

The nuts were crushed. The gray fatty mass was boiled with 1N potassium hydroxide solution for 60 min, neutralized with 1N HCl and extracted with ethyl acetate. The solution was dried over sodium sulfate, and the solvent was evaporated. The residue was melted at 70° C. and purified in a cellulose-packed column (at 70° C.). The extract contained approx. 50% tri-, di-, and monoglycerides and approx. 50% sterols, triterpene alcohols and esters thereof.

### EXAMPLE 2

### Lipophilic Extract From Kernels (Nuts) of Vitellaria paradoxa

The kernels were crushed, the mixture was heated and filtered while hot or extracted with hexane. The fraction was treated as raw shea butter. Using the standard methods of fat chemistry, it was deacidified, bleached and fractionated to shea stearin and shea olein. The shea olein fraction was dekaritinized, and successively washed, bleached, transesterified and hydrogenated with sodium hydroxide solution, sodium chloride solution and water. Repeat fractionation gave a product with approx. 50% unsaponifiable fraction. This extract was further stabilized with 0.1% β,δ-tocopherols.

The product contained approx. 25% triterpene alcohol esters and sterol esters, and approx. 2-5% cinnamic acid esters.

### EXAMPLE 3

A shea butter (karite butter) prepared and refined according to food standards, with a high triglyceride content, was used. The proportion of unsaponifiable components was 5-8%, in their turn consisting of 75% triterpene alcohols, 18% hydrocarbons, 5% sterols and 2% methyl sterols (iodine number: 55-71; peroxide number<5 mEq; melting point 32-40° C.). The product contained more than 40% of C18:0 fatty acid (stearic acid) and C18:1 fatty acids (mostly oleic acid).

### EXAMPLE 4

### Soft Capsules with Vitellaria Extract and Glucosamine

A soft capsule was prepared from approx. 270 mg of a vegetable capsule shell (tapioca starch, maltitol, sorbitol, glycerol, glycerol monostearate, red iron oxide) and filled with the following filling:

| | |
|---|---|
| Glucosamine sulfate 2 KCL | 400.00 mg |
| Triglyceride fish oil (30% EPA + DHA) | 200.00 mg |
| Vitellaria extract (example 2) | 100.00 mg |
| Lecithin (60% phosphatides) | 20.00 mg |
| (filling weight 720 mg) | |

After mixing, milling (homogenizing) and deaerating, the filling was filled in soft gelatin capsules at approx. 30° C. The capsule was immediately cooled rapidly with air (10° C.) for approx. 5 min and dried in the air stream (20° C., 20% RH).

### EXAMPLE 5

### Soft Capsules with Vitellaria Extract, Glucosamine and Chondroitin

A soft capsule was made from approx. 500 mg of a soft gelatin capsule shell (gelatin, glycerol, water, brown iron oxide) and filled with the following filling:

| | | |
|---|---|---|
| Glucosamine sulfate 2 KCL | 667.00 | mg |
| Chondroitin sulfate | 40.00 | mg |
| Fish oil, ethyl ester form with 55% EPA + DHA | 300.00 | mg |
| Vitellaria extract (example 3) | 450.00 | mg |
| Lecithin (60% phosphatides) | 20.00 | mg |
| (filling weight 1477 mg) | | |

After mixing, milling (homogenizing) and deaerating, the filling was filled at approx. 30° C. in soft gelatin capsules. The capsule was immediately cooled rapidly with air (10° C.) for approx. 5 min and dried in the air stream (20° C., 20% RH).

### EXAMPLE 6

### Soft Capsules with Glucosamine and Shea Butter

A soft capsule was made from approx. 270 mg of a vegetable capsule shell (tapioca starch, maltitol, sorbitol, glycerol, glycerol monostearate, red iron oxide) and filled with the following filling:

| | |
|---|---|
| Glucosamine sulfate 2 KCL | 400.00 mg |
| Vitellaria extract (example 1) | 300.00 mg |
| Tween 80 | 4.00 mg |
| (filling weight 704 mg) | |

After mixing, milling (homogenizing) and deaerating, the filling was filled in the capsules at approx. 40° C.

### EXAMPLE 7

### Soft Capsules with Glucosamine and Shea Butter

A soft capsule was made from approx. 200 mg of a soft gelatin capsule shell (gelatin, glycerol, sorbitol, water, brown iron oxide) and filled with the following filling:

| | |
|---|---|
| Glucosamine sulfate 2 KCL | 350.00 mg |
| Vitellaria extract (example 1) | 190.00 mg |
| Capmul MCM C8 | 40.00 mg |
| (caprylic acid monoglyceride) | |
| (Abitec Corp.) | |
| Tween 80 | 20.00 mg |
| (filling weight 600 mg) | |

After mixing, milling (homogenizing) and deaerating, the filling was filled in the capsules at approx. 30° C. The capsule was immediately cooled rapidly with air (10° C.) for approx. 5 min and dried in the air stream (20° C., 20% RH).

In a release apparatus according to USP/EP (paddle, 900 ml, 0.1N HCl, 100 rev/min), starting from 15 min (opening of the capsules) the capsules formed an emulsion that was stable (did not cream) for 1 h.

1.-16. (canceled)

17. A composition containing a lipophilic extract from Vitellaria paradoxa and at least one amino sugar or a pharmaceutically acceptable salt thereof.

18. The composition according to claim 17, wherein said composition additionally contains a hydrophilic extract from Vitellaria paradoxa.

19. The composition according to claim 17, wherein said at least one amino sugar is selected from the group consisting of D-glucosamine, N-acetylglucosamine, hyaluronic acid, D-galactosamine, N-acetylgalactosamine, chondroitin, dermatan, keratan, heparin and pharmaceutically acceptable salts thereof.

20. The composition according to claim 17, wherein said lipophilic Vitellaria extract contains at least 50% triglycerides or at least 50% triterpene alcohol esters.

21. The composition according to claim 17, wherein said extract is obtained from Vitellaria species selected from the group consisting of Vitellaria Gaertn.f., Vitellaria campechiana, Vitellaria mammosa (L.), Vitellaria nervosa (DC.), Vitellaria paradoxa Gaertn., and Vitellaria paradoxa subsp. Nilotica.

22. The composition according to claim 17, wherein said composition is a solidified melt at room temperature, which becomes pastelike at a temperature above 35° C.

23. The composition according to claim 17, wherein said Vitellaria extract has a solidification or melting range from 20 to 45° C.

24. The composition according to claim 17, wherein said Vitellaria extract has a drop point from 25 to 40° C.

25. A capsule, containing a composition according to claim 17, as filling.

26. The capsule according to claim 25, wherein said capsule is a soft capsule.

27. The capsule according to claim 25, wherein said capsule forms an emulsion on contact with the gastric juice.

28. A method of production of a capsule according to claim 25, wherein said capsule is filled with a composition containing a lipophilic extract from Vitellaria paradoxa and at least one amino sugar or a pharmaceutically acceptable salt thereof.

29. The method according to claim 28, wherein said capsule capsule is produced in the rotary die process.

30. Method of use of a composition according to claim 17 for the production of a capsule, said method comprising the step of filling a capsule with said composition.

31. Method of use of a lipophilic Vitellaria extract for the production of a pharmaceutical or dietetic capsule preparation containing an amino sugar for the prophylaxis and treatment of rheumatic diseases, said method comprising the step of preparing a composition by blending said lipophilic Vitellaria extract with an amino sugar.

32. Method of use of a capsule according to claim 25 for the prophylaxis and treatment of rheumatic diseases, said method comprising the step of preparing said capsule by filling a capsule material with a composition containing a lipophilic extract from Vitellaria paradoxa and at least one amino sugar or a pharmaceutically acceptable salt thereof.
